(19) United States
(12) Patent Application Publication
(10) Pub. No.: US 2023/0103774 A1
(43) Pub. Date: Apr. 6, 2023

(54) USE OF KAURANE COMPOUNDS IN PREPARATION OF DRUG FOR PREVENTION AND TREATMENT OF SEPSIS AND MULTIPLE ORGAN DAMAGE

(71) Applicant: KEY-PHARMA BIOMEDICAL INC., Dongguan, Guangdong (CN)

(72) Inventors: Shanping Wang, Dongguan, Guangdong (CN); Wen TAN, Dongguan, Guangdong (CN)

(21) Appl. No.: 17/799,683

(22) PCT Filed: Apr. 12, 2021
(86) PCT No.: PCT/CN2021/086531

§ 371 Date: Sep. 28, 2022

(30) Foreign Application Priority Data
Feb. 15, 2020 (CN) 202010094166.X

(51) Int. Cl. A61K 31/19; A61P 31/00; A61P 9/04; A61P 11/00; A61P 1/16; A61P 13/12; A61P 37/02

(52) U.S. Cl. CPC A61K 31/19; A61P 31/00; A61P 9/04; A61P 11/00; A61P 1/16; A61P 13/12; A61P 37/02

(57) ABSTRACT

The invention discloses the medicinal use of kaurene compound in the prevention and treatment of sepsis, systemic inflammatory response syndrome (SIRS) and multiple organ failure caused by sepsis, including acute pulmonary failure, acute heart failure and renal failure.

### TECHNICAL FIELD

The invention discloses the relieving and therapeutic effects of kaurene compounds in sepsis, systemic inflammatory response syndrome (SIRS) and multiple organ failure caused by sepsis. The invention discloses that the kaurene compounds can significantly reduce the mortality caused by sepsis; It can reduce the inflammatory response caused by sepsis, inhibit the proliferation of inflammatory cells caused by sepsis, and inhibit the increased level of cytokines in plasma. The invention discloses that the kaurene compounds have significant relieving and therapeutic effects on acute respiratory distress syndrome caused by sepsis, and lung injury, myocardial injury, heart failure, liver injury, kidney injury and spleen injury caused by sepsis. At the same time, it can also alleviate and treat remodeling and fibrosis of the above-mentioned organs after recovery from sepsis. The invention discloses the inhibition and regulation effects of the kaurene compounds on the activation and proliferation of macrophages when sepsis occurs.

### BACKGROUND OF THE INVENTION

Sepsis is a dysregulated host immune response to infection, and then results in multiple organ dysfunction (heart, liver, spleen, kidneys, and lungs). In clinical practice, sepsis could induce multiple organ dysfunction, including cardiovascular system (including microcirculatory system), respiratory system, renal system, nervous system, blood system and liver system. It has two vital stages during the pathology of sepsis: systemic inflammatory response and compensatory anti-inflammatory response. The balance between these two has an important role in the survival and prognosis of patients. The mortality of sepsis is up to 30%, and it is one of the main leading death in clinic.

Pathogens, like the lipopolysaccharide (LPS) from gram-negative bacteria, bacterial lipoprotein, viral double or single stranded nucleic acid, etc., can induce sepsis through pathogen associated molecular patterns (PAMPs). Pathogens can be recognized by pattern recognition receptors (PRRs) on the surface of human innate immune cells, such as Toll-like receptors (TLRs). When combined, it can initiate and activate host inflammatory immune response. For example, LPS can activate the TLR4 receptor on the surface of macrophages, and then activate NF-κB and MAKP and other inflammatory transcription factors, thereby producing a series of large amounts of inflammatory factors such as TNF-α, IL-1β, IL-6 and related inflammatory proteins, resulting in excessive inflammatory response and recruiting more inflammatory cells via chemotactic response, and then resulting in a vicious cycle and form a vicious circle. This eventually leads to an excessive systemic inflammatory immune response, and in severe cases, (leads to) systemic inflammatory response syndrome (SIRS) or sepsis and multiple organ failure, and even death (Qiu P. et al., Inflammation. 2019:42(1): 6-19). Non-infectious diseases such as heart failure, cerebral infarction, severe trauma, and extensive burns may also induce systemic inflammatory response syndrome. Sepsis (SIRS combined with infections), with a mortality of up to 30%, is one of the leading death in clinic. The lung is the most vulnerable organ easily affected by sepsis. About 70% of sepsis patients will develop acute lung injury, acute respiratory depression syndrome (ARDS) and acute respiratory failure successively. Similarly, kidney failure, liver failure and heart failure can also happen. Single or multiple organ failure eventually leads to death in septic patients. When survived, patients may suffer permanent organ damage because of fibrosis.

Macrophages have been found to play a key role in the progression of sepsis. Macrophages are the primary cells responsible for the production of key pro-inflammatory cytokines. Static macrophages in immune homeostasis possess dominate by oxidative phosphorylation. During sepsis, macrophages are activated and release a large number of inflammatory factors and chemical toxic substances, such as IFN-γ, IL-1, IL-6, I-L-12, IL-13, IL-1β, IL-10, IL-23, IL-17, TNF-α, Nitric oxide (NO), and peroxides (ROS). In turn, these can activate NK cells and lymphocytes, leading to increased release of cytokines, local and systemic inflammatory responses, causing “cytokine storm”.

Activated macrophages undergo profound metabolic changes and remodeling, and increase ATP consumption (Kelly B. et al., Cell Res. 2015:25 (7): 771-784.). Activated macrophages can be divided into M1 and M2 macrophages according to different cytokines that induce activation and different metabolic pathways in mitochondria. Inhibition of glycolysis in macrophage reduces the production of inflammatory factors, thereby regulating macrophage function. Therefore, the regulation of immune response may also be achieved through the metabolic regulation and remodeling of macrophages, thus interfering with in the occurrence and development of sepsis.

Currently no specific treatment for sepsis. In clinic, the strategies for the treatment of sepsis are removing the infection tissue, initial fluid resuscitation, anti-hypotensive shock, ventilation, and adjunctive antibiotic therapy. It is still blurred that immunomodulatory therapy, like anti-inflammatory factor agents have a definitive therapeutic effect for sepsis. In addition, corticosteroid therapy is widely used, but the use of systemic steroids in sepsis is still controversial due to its clinical efficacy and potential side effects. Moreover, the morbidity and mortality of sepsis show no significant slowdown over the past few decades. Sepsis is still a major life-threatening disease. And its mortality rate has declined in recent years, but the morbidity of sepsis is rising high. The World Health Organization also decided to raise the awareness of sepsis and called on countries and health institutions to promote the diagnosis and treatment of sepsis. Therefore, the development of safe and effective therapeutic drugs for sepsis and multiple organ failure is urgently needed for septic patients.

Compound A is a beylane terpenoid isolated from stevia. Stevia is a well-known traditional plant in South America and is a widely used sweetener worldwide. The effects of stevia on the metabolic and cardiovascular system have also been reported (Geuns JMC. Stevioside. Phytochemistry. 2003; 64(5):913-21).

Previous studies have shown that kaurane-like compounds, represented by compound A, have protective effects on heart and brain and can be used to treat myocardial ischemia and cerebral infarction (Patent 1: CN100508962 C). In addition, compound A and related kaurene type compounds also inhibit inflammation caused by tissue damage and inhibit fibrosis in cardiac and lung tissues (Patent 2: CN1083484881 A). Compound A can also be used for metabolic diseases and diabetic myocarditis. It was also proved that compound A also has the inhibitory effect on some inflammatory cytokines, such as TNF-α and interleukin 6, etc.

However, compound A and related kaurene compounds have not been reported in the treatment of sepsis caused by pathogenic infection and the protection of organ failure caused by sepsis. It is now known that the cytokine storm and immune dysfunction caused by infection or other severe diseases are the main causes of sepsis, and the excessive activation of macrophages plays a key role in the initiation and development of the above cytokine storm. However, the effect of compound A and related kaurene compounds on immune dysfunction and macrophage activation induced by the above-mentioned infection has not been reported either.

In this invention, we disclose for the first time that compound A and related kaurene compounds can be used to treat sepsis and multiple organ damage caused by sepsis. It can alleviate heart, liver, spleen, lung and kidney dysfunction caused by sepsis and reduce the mortality of septic mice. Compound A and related kaurene compounds can also inhibit the polarization of macrophages caused by pathogen infection and sepsis, and inhibit the expression of various cytokines and chemical toxic substances, so as to regulate the abnormal immune inflammatory response caused by infection and achieve the effect of treating sepsis.

### DETAILED DESCRIPTION OF THE INVENTION

The purpose of the invention is to provide kaurene compounds in the preparation of medicament for the treatment of prevention of sepsis and sepsis-induced multi-organ injury. The invention discloses a novel drug for treating and/or preventing multiple organ damage in sepsis.

The present invention discloses kaurene compounds, such as compound A (structural formula (I)), for treating sepsis and multiple organ failure. The structural formula (I) represents a class of natural, synthetic, or semi-synthetic compounds. Many of these compounds are known to the public (Kinghorn A D, 2002, P86-137; Sinder B B et al., 1998; Chang F R et al., 1998; Hsu F L et al., 2002). Compounds of structural formula (I) may have one or more asymmetric centers or may exist as different stereoisomers.

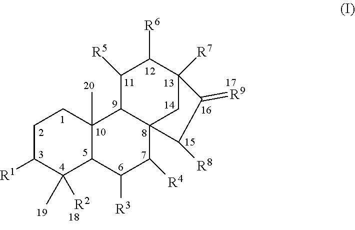

wherein ii. R1: Hydrogen, hydroxyl,
or alkoxy. iii. R2:
Carboxyl, carboxylate, acyl halide, aldehyde, hydroxymethyl, and ester, acrylamide, acyl, or ether groups that can form carboxyl. iv. R3, R4, R5, R6, R8: Oxygen, hydroxyl, hydroxymethyl, and ester or alkoxymethyl groups that hydrolyze to hydroxyl.
v. R7: Methyl, hydroxyl, and ester or alkoxymethyl groups that hydrolyze to hydroxymethyl. vi. R9: Methylene or oxygen.

The preferred compound A is shown as structural formula (I′). The compound has a kauri structure, is substituted at the C13 position and derivatized at C17 and C18. The compound may have multiple asymmetric centers and have different stereoisomers or diastereoisomers. The absolute configurations of positions 8 and 13 are either (8R, 13S) or (8S, 13R).

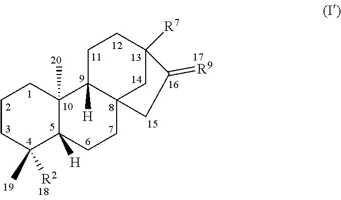

Therein vii. R2: Carboxylate, carboxylate, aldehyde group, hydroxymethyl, methyl ester, acyl methyl, acyl halide. viii. R7: Methyl, hydroxymethyl or methyl ether. ix. R9: Methylene or oxygen.

Compound A can be obtained after the hydrolysis of natural stevioside. Compound B is the glycoside ligand of stevioside, and steviol is the glycoside of compound B. Compound A and B are isomers. Compound B can be obtained by the hydrolysis and oxidation of stevia, or by the catalytic reaction of animal intestinal bacteria.

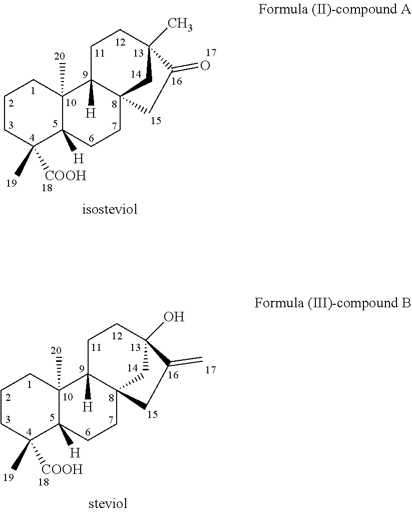

The molecular formula of compound A is C20H30O3 and the chemical name is (4α, 8β, 13 β)-13-METHYL-16-OXO-17-Norkauran-18-OIC acid. Compound A is also known as ENT-16-ketobeyran-18-Oic acid. This compound is a tetracyclic diterpenoid containing kaurene structure, in which the absolute configuration of asymmetric carbon atoms is (4R, 5S, 8R, 9R, 10s, 13s), with methyl substituents on C 13, carbonyl group on C 16, and carboxyl group on C 18 (Rodrigues et al., 1988).

The molecular formula of compound B is C20H30O3 and the chemical name is ENT13Hyrdoxyaur-16-EN-18-Oic acid, it is also known as steviol. The compound is also a tetracyclic diterpenoid containing kaurene structure. Among them, the absolute configuration of chiral carbon atoms is (4R, 5S, 8R, 9R, 10S, 13S), with hydroxyl group substituents on C 13, methylene group adjacent to C 16, and carboxyl group on C 18 (Rodrigues et al., 1993).

Compounds A or B may also exist as carboxylate at C 18, wherein the carboxylate are sodium, basic metal, chloride or halogen. Both compounds A and B have kaurane structure. Compound A is the preferred compound of the present invention. The invention discloses that compound A or B has similar therapeutic effects in the treatment and prevention of cardiac hypertrophy and pulmonary hypertension. It can be inferred that all other compounds of structural formula (I) also have the same therapeutic effect as compound A. Compound B has been reported to be mutagenic under certain conditions in vitro. Therefore, compound A is more suitable as a therapeutic agent than compound B. Compound A used in the present invention is the sodium salt of compound A with better solubility.

The present invention discloses the application of compound A (structural formula (I)) in treating and preventing sepsis or acute systemic inflammatory response syndrome (SIRS) caused by infection. After LPS injection, all mice died of sepsis. When intraperitoneally administered with compound A, the death of septic mice was significantly reduced after LPS injection. In an experiment of this invention, all mice died within 24 h after intraperitoneal injection of a large dose of LPS. When treated with compound A (10-20 mg/kg), the survival rate of mice was significantly improved, with up to more than two-thirds of septic mice surviving. The survival rate of septic mice was significantly correlated with the dose of Compound A.

The invention also discloses that the white blood cells, neutrophils and monocytes in LPS induced septic mice are significantly increased when compared with the Control group, indicating that mice in the LPS-induced group exhibited inflammatory response; when mice treated with compound A, the increased level of white blood cells, neutrophils, and monocytes were significantly reduced than those of LPS group and are no significant difference with the Control group. These results indicated that compound A could significantly regulate the immune dysfunction caused by sepsis or SIRS and restore it to normal. In another embodiment of the invention disclosed, compound B in structural formula (I) also has a similar effect to that of compound A. The above disclosures have not been previously reported, nor can it be predicted or inferred by industry insiders, and should be regarded as novel and creative.

On the other hand, the present invention also disclosed a surprising finding that leukocytes, neutrophils, and monocytes increased after dexamethasone was administered to LPS-induced septic mice, indicating that dexamethasone has no beneficial effect in regulating immune dysfunction, and even worse. In addition, spleen is an important organ for regulating immune response. Immunoglobulins produced by spleen are crucial for fighting against pathogens. The spleen/body weight ratio increases significantly after LPS infection. However, this proportion was significantly reduced when dexamethasone administration and even lower than that of the Control group, revealing an adverse effect of hormone. These results may explain why corticosteroids fail in clinical practice. The invention discloses for the first time that the use of compound A to treat sepsis can avoid the toxic side effects of corticosteroids in clinic. In another embodiment of the invention disclosed, compound B in structural formula (I) also has a similar effect to that of compound A. The above disclosure has not been previously reported, nor can it be predicted or inferred by industry insiders, and should be regarded as novel and creative.

Cytokines (cytokine storm) caused by pathogen infection is essential for the progression of sepsis. In the experiment disclosed by the invention, pro-inflammatory cytokine (TNF-α, IL-6 and IL-1β) expression in plasma was significantly increased (P<0.01) in the LPS group (versus the controls) after 6 h intraperitoneal injection of LPS. When administered of different doses of compound A, the increased TNF-α, IL-1β and IL-6 in plasma were dose-dependently decreased. It has been reported that compound A can inhibit the increase of these inflammatory factors in ischemic injury. The present invention discloses for the first time that compound A can inhibit the increase of cytokines caused by sepsis. The pathological mechanism of sepsis with ischemia are completely different. Compound B in structural formula (I) in another experiment disclosed by the invention also has a similar effect with compound A. The above disclosures have not been previously reported, nor can it be predicted or inferred by industry insiders, and should be regarded as novel and creative.

Sepsis can result in multi-organ failure. The lung is the most vulnerable organ easily affected by sepsis. In the experiments of this invention, in septic lung tissues, we found various grades of lung tissue hemorrhage or consolidation, alveolar septal thickening, infiltration of inflammatory cells, and narrowing of the lumen. When administered with compound A, lung inflammation was significantly reduced, lung tissue structure was basically normal, lung function was significantly recovered, blood oxygen saturation was significantly increased, and airway reactivity returned to normal in septic mice. The invention discloses for the first time the significant efficacy of compound A in the treatment of acute lung injury and failure caused by sepsis, which has never been reported before. The invention also discloses that compound B in structural formula (I) also has similar results with compound A. Existing literature has reported the protective effects of compound A and compound B in the treatment of pulmonary injury caused by aortic hypertension or bleomycin-induced. The present invention is the first to report the significant effect of compound A for the treatment of lung injury and failure caused by sepsis or acute systemic inflammatory response syndrome (SIRS) caused by pathogen infection. Bleomycin-induced or aortic hypertension is fundamentally different from infection and SIRS in etiology, and its pathogenesis is also different. Therefore, it can not be predicted or inferred by industry insiders from the existing literature. The invention shall be deemed to be novel and creative.

The invention also discloses the role of compound A for the treatment of reduced cardiac function and heart failure and arrhythmia caused by sepsis. When compared to controls, EF, FS, SV and CO were decreased by 2.27-, 2.56-, 2.44- and 1.89-fold, respectively, in septic mice. Mice pretreated with STV-Na exhibited markedly increased EF, FS, SV and CO versus the LPS group, indicating that STV-Na improved cardiac function in septic mice. In addition, biochemical analysis demonstrated that the levels of lactate dehydrogenase (LDH), as well as creatine kinase (CK), in plasma were substantially upregulated in LPS versus the controls. Similarly, STV-Na pretreatment significantly decreased LDH and CK expression in plasma. Taken together, these data indicate that STV-Na could improve the cardiac function of mice with LPS-induced sepsis.

Superoxide (ROS) is an important chemical agent that causes tissue damage. In another experiment disclosed by the invention, the level of ROS in heart of septic mice was fold increased than that of the Control group. When treated with compound A, the increased level of ROS was inhibited. In addition, macrophages play an important role in mediating immune inflammatory responses. The present invention also discloses that macrophage M1 and M2 in cardiac tissue are significantly increased during sepsis, and the levels of macrophage M1 biomarkers (IL-1β, McP1 and CD11c) and M2 macrophage biomarkers (Ym1 and CD206) are significantly elevated than those of the Control. The biomarkers of M1 and M2 macrophages and the number of macrophages were significantly decreased after the administration of compound A. Compound A can regulate the inflammatory response of heart tissue induced by macrophages by inhibiting M1 and M2 macrophages.

The invention discloses that compound A can treat sepsis induced heart injury and heart failure by inhibiting the production of superoxide (ROS) and inhibiting macrophages proliferation and activation. Existing literature has reported the effect of compound A on the treatment of myocardial injury and cardiac failure caused by ischemia or aortic hypertension. The present invention discloses the significant effect of compound A on the treatment of heart injury and heart failure caused by sepsis caused by pathogen infection or acute systemic inflammatory response syndrome (SIRS) for the first time. Ischemia or aortic hypertension is fundamentally different from infection and SIRS in etiology, and its pathogenesis is also different. Therefore, it is impossible for people in the industry to infer from the existing literature that Compound A can protect myocardial injury and heart failure caused by sepsis or SIRS. The present invention should be regarded as novel and inventive. In another embodiment disclosed in the present invention, the compound B in the structural formula (I) also has a similar effect to that of the compound A. The above disclosures have not been reported in the past, nor can people in the industry predict or infer, and should be regarded as novel and creative.

The invention discloses the effect of compound A on renal function damage and failure in the treatment of sepsis. Plasma creatinine (CREA) and urea nitrogen (BUN) are mainly eliminated by the kidneys, so changes in plasma creatinine (CREA) and urea nitrogen (BUN) concentrations can reflect renal function. In septic animals, plasma creatinine (CREA) and blood urea nitrogen (BUN) concentrations are nearly doubled increase almost exponentially, renal function is markedly decreased, and eventually fails. From histopathological staining, it can be seen that the glomerular volume of septic animals increases, inflammatory cell infiltration can be seen in the renal interstitium, renal tubules are widely expanded, epithelial cells appear edema, and vacuolar degeneration can be seen. After administration of compound A, the plasma creatinine (CREA) and urea nitrogen (BUN) concentrations of the septic animals were significantly reduced and almost returned to normal levels; some lesions appeared in the glomerulus and renal tubules, and the lesions were milder than those in the model group, indicates significant improvement in kidney function. However, after administration of corticosteroids, creatinine (CREA) in septic animals did not change significantly. Compound A has a better protective effect on renal function in animals with sepsis compared with corticosteroids. The present invention discloses for the first time the therapeutic effect of compound A in protecting sepsis caused by pathogenic infection or renal failure caused by SIRS. In another embodiment disclosed in the present invention, the compound B in the structural formula (I) also has a similar effect to that of the compound A. The above disclosures have not been reported in the past, nor can anyone in the industry predict or infer, and should be regarded as novel and creative. The present invention discloses the effect of compound A in the treatment of sepsis with liver damage. Impairment of liver function usually manifests as increased plasma alanine aminotransferase and aspartate aminotransferase from the liver. In septic animals, alanine aminotransferase and aspartate aminotransferase were significantly elevated, suggesting liver damage. From histopathological staining, it can be seen that the central vein hepatocytes of septic animals are swollen and degenerated, with multiple necrosis and a large number of inflammatory cells infiltration. After administration of compound A, the above-mentioned transaminases were significantly decreased, the infiltration of inflammatory cells was reduced, and the recovery was close to normal. The present invention discloses for the first time the therapeutic effect of compound A in protecting liver failure caused by sepsis or SIRS caused by pathogenic infection. In another embodiment disclosed in the present invention, the compound B in the structural formula (I) also has a similar effect to that of the compound A. The above disclosures have not been reported in the past, nor can anyone in the industry predict or infer, and should be regarded as novel and creative.

The present invention discloses the effect of compound A in preventing and treating fibrosis of affected organs after recovery from sepsis. Animals that survived sepsis developed significant fibrosis and collagen deposition in the lungs, heart, liver, and kidney, whereas compound A treated septic animals showed a marked reduction in these fibrosis. Existing literature reports compound A in the treatment of myocardial or lung tissue fibrosis or remodeling caused by high aortic pressure or ischemia. However, the etiology and pathological mechanism of high aortic pressure or ischemia are completely different from those of sepsis or SIRS. The present invention discloses for the first time the therapeutic effect of compound A in protecting organs caused by pathogenic infection caused by sepsis or SIRS, including fibrosis and remodeling of the lung, heart, liver and kidney, etc. In another embodiment disclosed in the present invention, the compound B in the structural formula (I) also has a similar effect to that of the compound A. The above disclosures have not been reported in the past, nor can anyone in the industry predict or infer, and should be regarded as novel and creative.

Activation of macrophages is a critical step in the development and progression of sepsis. The invention discloses the effect of compound A in inhibiting and regulating the activation and polarization of macrophages in sepsis. It is disclosed that compound A achieves alleviation and treatment of sepsis by modulating the activation and polarization of macrophages. M1 macrophages are classically activated macrophages, usually activated by LPS and or IFN-γ. After activation, they release pro-inflammatory factors such as TNF-α and IL-6, as well as NO and ROS, etc. While killing pathogenic bacteria, it induces the production of inflammatory factors and triggers septic shock, which is the main factor of early death in sepsis. The M2 type is often stimulated by IL-4 and or IL-13, it secretes IL-10, and highly expresses CD206, Ym1, Fizz1, ARG1, etc. The polarization of M2 macrophages is involved in the occurrence and development of many diseases, and the whole process of many chronic inflammatory diseases involves the participation of M1/M2 cells. The invention discloses that in LPS-induced sepsis, compound A can significantly inhibit M1 and M2 macrophages in the peritoneal cavity of mice, thereby reducing the secretion of early proinflammatory cytokines.

Mitochondria are dynamic organelles required to maintain normal physiology. Pathogen associated molecules (PAMPs) such as LPS can cause mitochondrial damage, generate excess ROS, increase mitochondrial membrane permeability, decrease membrane potential, and release a large amount of cytochrome C and further exacerbated cellular damage and inflammatory responses. The invention discloses that administration of compound A during sepsis can significantly protect mitochondria, increase mitochondrial membrane potential, reduce cytochrome C release; increase the number of mitochondria, reduce the production of superoxide such as NO in cells, thereby inhibiting inflammation cytokine TNF-α and IL-1β. The invention also discloses that activator A plays a therapeutic and protective effect on sepsis by restoring mitochondrial function.

The invention discloses that compound A exerts its effects of inhibiting and regulating macrophages by inhibiting the expression of HIF-1α. After LPS injection, the HIF-1α expression was increased. When administered with compound A, the increased level reduced. When we inhibited HIF-1α expression with DMOG, a competitive inhibitor of HIF-1α prolyl hydroxylase, the metabolic regulation effect of Compound A on macrophages was abolished. In another implementation of the present disclosure, the compound B in the structural formula (I) also has a similar effect to that of the compound A. The content of the above disclosure about the regulatory effect of the compound of structural formula (I) on macrophages and its main mechanism of antisepsis as the compound of structural formula (I) has not been reported before, nor can anyone in the industry predict it. and inferred, shall be considered novel and inventive.

### BRIEF DESCRIPTION OF THE DRAWINGS

FIG. 1 is the survival curve of each group of mice after intraperitoneal injection of different doses of LPS in Example 1 of the present invention.

FIG. 2 is the effect of intraperitoneal injection of different doses of compound A on the mortality of LPS-induced septic Balb/C mice in Example 2 of the present invention.

FIG. 3 is the effect of Compound A on cardiac function of LPS-induced mice in Example 4 of the present invention.

FIG. 4 is the effect of compound A on plasma CK and LDH of LPS-induced septic mice in Example 5 of the present invention.

FIG. 5 is the effect of compound A on pulmonary function after compound A administration by whole body plethysmography recording (Penh, EF50, TV) in Example 6 of the present invention.

FIG. 6 is the effect of compound A on LPS-induced liver dysfunction in Example 7 of the present invention

FIG. 7 is the effect of compound A on LPS-induced renal dysfunction in Example 8 of the present invention

FIG. 8 is the effect of compound A on M1/M2 primary peritoneal macrophages of LPS induced septic mice in Example 9 of the present invention.

### EXAMPLES

The methods and examples of the present invention are provided in detail as follows.

Detail Methods

To further illustrate the technologies used to achieve the objects of the present invention, detailed methods, techniques, procedures and special features regarding in determining and identifying the pharmaceutical and therapeutic uses of the kaurene compounds in the present invention are described below. Examples provide experimental methods and results which are utilized for supporting the invention, and for validating the animal models used in the present invention. Proper control and statistical analysis are used in all the experiments in this invention. The following examples are provided to illustrate, not limit, the invention. The methods and techniques utilized to screen and to determine the therapeutic use of some kaurene compounds of formula (I). The therapeutic use of other compounds of formula (I) can also be determined in the same way.

The examples provided in the present invention are used to support the experimental methods and results of the present invention and to validate the animal models used in the present invention. Appropriate controls and statistical tests were used in all experiments of the present invention. The following examples are provided to illustrate, not limit, the invention. The methods and techniques utilized to screen and to determine the therapeutic use of some kaurene compounds of formula (I). The therapeutic use of other compounds of formula (I) can also be determined in the same way.

### Experimental Materials

Experimental animal: Adult male Balb/c mice, body weight 20 g±5 g, 6-8 weeks old. Mice are housed in acrylic cages with food and water ad libitum under an environmentally controlled condition.
Chemical reagent: Compound A (ent-17-norkaurane-16-oxo-18-oic acid, molecular formula, C20H40O3, molecular weight: 318.5) is obtained from stevioside through acid hydrolysis and crystallization purification. The sodium salt of compound A can be obtained by adding NaOH or other sodium-containing bases; the purity of the sodium salt of compound A measured by high performance liquid chromatography is greater than 99%.
Administration of test compound: intravenous or intraperitoneal injection or oral. Dosage; Compound A (or its sodium salt), 5 mg/kg to 60 mg/kg.

### Statistical Analysis

Differences between groups were compared by ANOVA (one-way ANOVA) and Fisher's test. P values for all tests were two-tailed, and P<0.05 was considered statistically significant.

### EXAMPLE 1

In this case, a mouse model of sepsis with intraperitoneal injection of LPS was established.

Forty-eight Balb/c mice (6-8 weeks, ♂) were randomly divided into 6 groups: the Control group, 10 mg/kg LPS group, 15 mg/kg LPS group, 20 mg/kg LPS group, 25 mg/kg LPS group. and 30 mg/kg LPS group, 8 mice per group. Mice were given intraperitoneal injection of LPS to establish a mouse model of sepsis. Different doses of LPS were given respectively, and the Control group was given the same volume of 0.9% saline to observe the changes in the survival rate of mice.

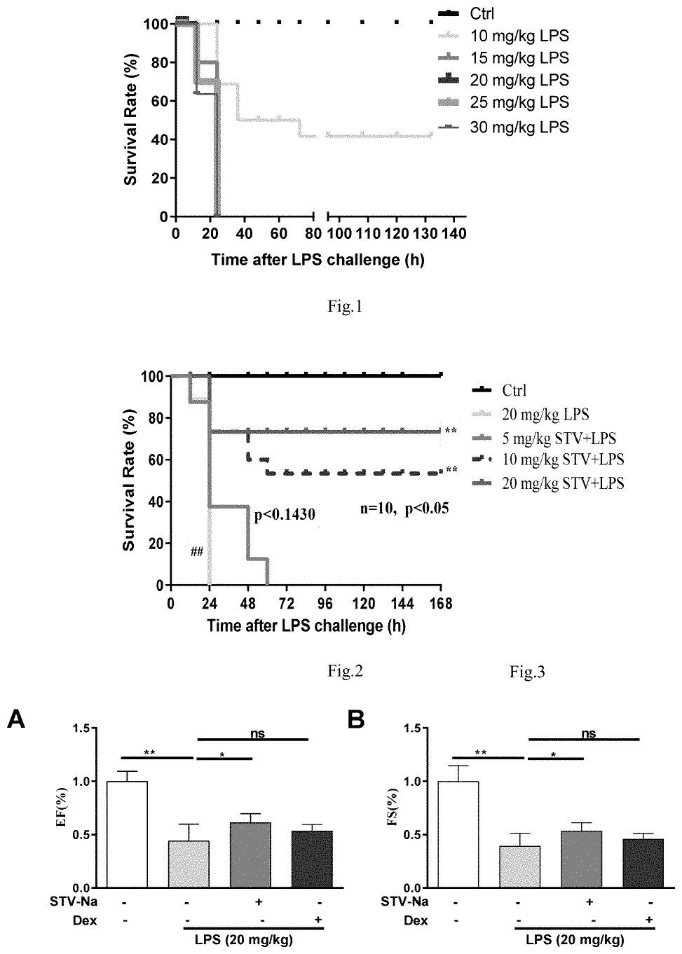

In this study, five different doses of 10 mg/kg, 15 mg/kg, 20 mg/kg, 25 mg/kg and 30 mg/kg were selected. In FIG. 1, 10 mg/kg is the median lethal dose, and 20-30 mg/kg is the lethal dose. To investigate the effect of Compound A on the survival rate of septic mice, we chose 20 mg/kg LPS to induce acute sepsis model. In the follow-up experiments, we chose intraperitoneal injection of 20 mg/kg LPS for 6 h as the lethal dose and modeling time of the mouse sepsis model.

### EXAMPLE 2

This example mainly observes the effect of intraperitoneal injection of different doses of Compound A on the mortality of LPS-septic Balb/C mice.

To observe the effect of intraperitoneal injection of different doses of compound A on the mortality of LPS-induced sepsis mice.

The mice were randomly divided into 6 groups:
(1) Control group: intraperitoneal injection of physiological saline (solvent) (0.1 ml/10 g) once a day for three consecutive days, 1 hour after intraperitoneal injection on the third day, intraperitoneal injection of physiological saline (0.2 ml/10 g);
(2) LPS group: intraperitoneal injection of saline (0.1 ml/10 g), 1 time/d for 3 consecutive days, 1 hour after intraperitoneal injection on the third day, intraperitoneal injection of LPS (20 mg/kg, 0.1 ml/10 g).
(3) Compound A (5 mg/kg)+LPS group: intraperitoneal injection of compound A (5 mg/kg, 0.1 ml/10 g), 1 time/d for 3 consecutive days, 1 hour after intraperitoneal injection on the third day, intraperitoneal injection LPS (20 mg/kg, 0.1 ml/10 g).
(4) Compound A (10 mg/kg)+LPS group: intraperitoneal injection of compound A (10 mg/kg, 0.1 ml/10 g), 1 time/d for 3 consecutive days, 1 hour after intraperitoneal injection on the third day, intraperitoneal injection LPS (20 mg/kg, 0.1 ml/10 g).
(5) Compound A (20 mg/kg)+LPS group: intraperitoneal injection of compound A (20 mg/kg, 0.1 ml/10 g), 1 time/d for 3 consecutive days, 1 hour after intraperitoneal injection on the third day, intraperitoneal injection LPS (20 mg/kg, 0.1 ml/10 g).
(6) Compound A (60 mg/kg)+LPS group: intraperitoneal injection of compound A (60 mg/kg, 0.1 ml/10 g), 1 time/d for 3 consecutive days, 1 hour after intraperitoneal injection on the third day, intraperitoneal injection LPS (20 mg/kg, 0.1 ml/10 g).

After intraperitoneal injection of LPS, the survival of mice in each group was observed every 12 h for six consecutive days.

As shown in FIG. 2, after intraperitoneal injection of 20 mg/kg LPS, all mice died within 24 hours, and 6 hours after intraperitoneal injection of LPS, all mice in the 5 mg/kg compound A group died, and there was no significant difference with the LPS group (p>0.05), while 10 mg/kg and 20 mg/kg of Compound A improved the survival rate of mice by 52% and 67%, respectively, and there was a significant difference between the two groups compared with the Control group (P<0.01), no death observed in the Control group at each time period. Based on the previously reports, the dose of 5 mg/kg for adults is an acceptable and safe dose, and the maximum blood concentration reaches 20 μM, which is equivalent to 45 mg/kg in mice. Accordingly, 10 mg/kg STV-Na was employed for further studies.

### EXAMPLE 3

This example mainly observes the general behavior of experimental mice in each group.

The fur color, consciousness, physical activity, diet, etc. of the mice were observed, and the heart, liver, spleen, lung and kidney of the mice were weighed 6 hours after intraperitoneal injection of LPS. In the Control group, there was no obvious abnormality in behavior, spirit, eating and fur color; After intraperitoneal injection of LPS, the mice in the other five groups gradually showed fatigue, lethargy, decreased appetite, fear of cold and unresponsiveness.

### EXAMPLE 4

This example mainly illustrates the effect of compound A in improving cardiac dysfunction in septic mice.

Six hours after intraperitoneal injection of LPS, mice in each group were weighed and anesthetized, and the cardiac function of the mice was detected by Vevo2100 imaging system. The mice were put into the recumbent position and fixed on a constant temperature heating plate under heart rate of 400-500 times per minute. The limbs were fixed on the four metal poles with tape. The ultrasound probe was placed in the left thoracic region of the mouse, and the short axis of the parasternal left ventricle was obtained by 2D ultrasound. The left ventricular motion was recorded at the papillary muscle level by M-ultrasound, and heart rate (HR), left ventricular systolic diameter (LVID: FS), left ventricular diastolic diameter (LVID: d), left ventricular systolic diameter (LVID: FS), left ventricular diastolic diameter (LVID: Fd), and left ventricular diastolic forearm thickness (LVPWF: FdF) were measured. Using Vevo2100 small animal ultrasound system software to process and analyze the acquired images. Determination of mouse plasma myocardial injury indexes (LDH and CK)

Ejection Fractions (EF), refers to the percentage of cardiac stroke volume in ventricular end-diastolic volume, which is an indicator of myocardial contractility. As shown in FIG. 3A, the echocardiography results showed that the ejection fraction of the LPS-induced septic mice was significantly lower than that of the Control group (p<0.01), indicating that intraperitoneal injection of LPS resulted in decreased cardiac contractility and decreased cardiac function. However, after compound A administration, the EF was significantly improved (p<0.05) compared with the LPS group, while the effect of dexamethasone was not significant. The left ventricular short-axis shortening rate (fractional shortening, FS) refers to the ratio of the inner diameter of the ventricle at the end systole to the inner diameter of the ventricular end diastole, and also reflects the systolic function of the heart (FS=(left ventricular end-diastolic diameter (LVDd)−Left ventricular end-systolic diameter (LVDs))/left ventricular end-diastolic diameter×100%). As shown in FIG. 3B, the FS of the LPS-induced septic mice was significantly lower than that of the Control group (p<0.01), while the FS of the compound A group was significantly higher than that of the LPS group (p<0.05), indicating that the cardiac systolic function of LPS-induced septic mice decreased, whereas that of the Dex group was not significant.

In conclusion, intraperitoneal injection of LPS can decrease the left ventricular ejection fraction and fractional shortening, and induce a certain degree of cardiac dysfunction. After treated with compound A, the cardiac function was improved, which could alleviate the cardiac dysfunction caused by LPS.

### EXAMPLE 5

This example mainly illustrates the effect of compound A in improving cardiac dysfunction in sepsis.

Serum lactate dehydrogenase (LDH) and creatine kinase (CK) were determined according to the istructions provided by Nanjing Jiancheng Biotechnology Co., Ltd.

Lactate dehydrogenase (LDH) activity in plasma (FIG. 4) LDH is an important glycolytic enzyme in human energy metabolism. When myocardial injury such as viral and rheumatic myocardial inflammation occurs, the level of plasma LDH increases, so it can be used as an effective indicator for the diagnosis of myocardial injury. As shown in FIG. 4A, the plasma LDH level of the LPS-induced septic mice was significantly higher than that of the Control group (P<0.01). After treated with compound A and Dex, the LDH levels in the plasma showed a downward trend, which was statistically significant when compared with the LPS group (p<0.01). Taken together, these data indicate that Compound A and Dex could ameliorate the myocardial injury of mice with LPS-induced sepsis.

Creatine kinase (CK) activity in plasma (FIG. 4B): In general, myocardial tissue CK is present in cardiomyocytes, and when blood levels of CK are elevated, it usually indicates or is developing myocardial damage. As shown in FIG. 4B, the plasma CK level in the LPS group was significantly higher than that in the control group (P<0.01). After compound A and Dex administration, the level of serum CK was significantly decreased with statistical significance (p<0.01) when compared with the LPS group, indicating that Compound A and Dex can alleviate LPS-induced myocardial injury in mice and achieve therapeutic effect.

### EXAMPLE 6

This example illustrates the effect of compound A in improving lung function in septic mice.

The lung function of each group of mice was measured at 6 h. Mice were placed in a closed chamber of a whole body plethysmograph (BUXCO, USA), and the plethysmographic chamber was connected to a sensor. As the animal breathes, the rise and fall of the chest changes the volume inside the chamber, and pressure transducers and amplifiers convert this volume change into an electrical signal, After processing, the respiration curve is displayed on screen, and the graphics are processed by relevant software to calculate the tidal volume (TV), the expiratory flow at 50% tidal volume (50% tidal volume expiratory flow, EF50), airway responsiveness (Penh), etc.

In this experiment, we used Buxco's pulmonary function testing system to measure and record the 50% tidal expiratory volume, tidal volume, and changes in airway responsiveness in each group of mice by whole body plethysmography to assess lung function. This method offers a real-time lung function of mice under non-invasive conditions. Penh is a pulmonary function parameter related to airway resistance and intrapleural pressure, and Penh is used as an index to evaluate airway response. It can be seen from FIG. 5 that 6 hours after LPS injection, the Penh of the LPS group was significantly higher than that of the Control group (p<0.01), and the EF50 and TV values were significantly lower than those of the Control group (p<0.01), indicating that the LPS-induced lung injury mice exhibited obvious airway hyperresponsiveness. However, EF50 and TV were significantly higher than those of the LPS group (p<0.01), indicating that compound A can ameliorate LPS-induced lung injury.

### EXAMPLE 7

This example illustrates the role of compound A in sepsis-induced liver damage.

The prepared plasma of each group was taken to determine the levels of alanine aminotransferase (ALT), aspartate aminotransferase (AST) and superoxide dismutase (SOD) in plasma by a microplate reader according to the manufacture's instruction.

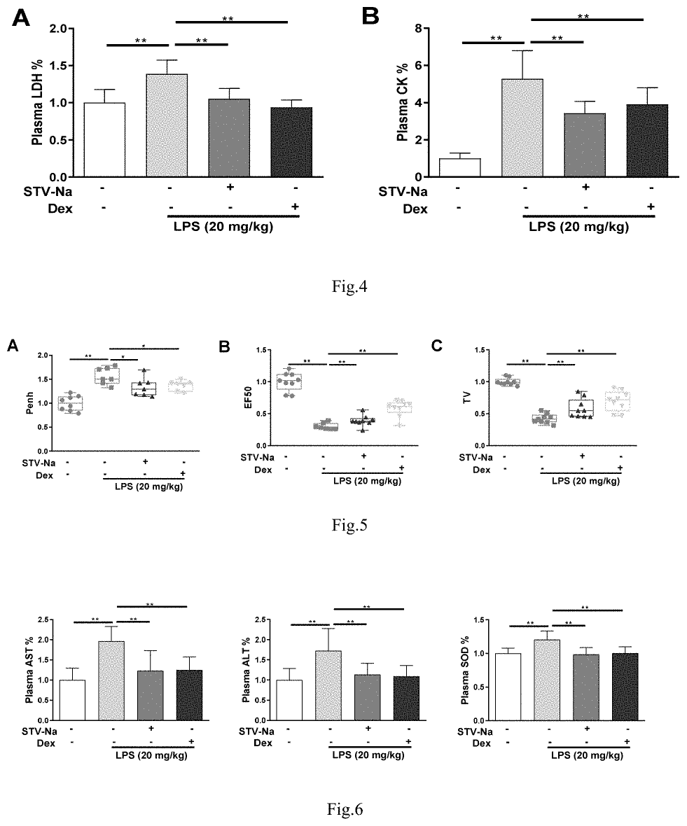

In order to observe the effect of compound A on the liver function of LPS-induced systemic inflammatory response in mice, we detected the changes of plasma ALT, ALT and SOD in mice, respectively. As shown in FIG. 6, the changes of ALT, ALT and SOD in plasma of mice were significantly decreased after compound A and Dex treatment when compared with the Control group (P<0.01), indicating that compound A can ameliorate liver function disorder of LPSinduced septic mice.

### EXAMPLE 8

This example illustrates the effect of Compound A in improving renal impairment in sepsis.

The prepared plasma of each group was taken to determine the levels of urea nitrogen (BUN) and creatinine (Crea) in plasma by a microplate reader following the manufacture's instruction.

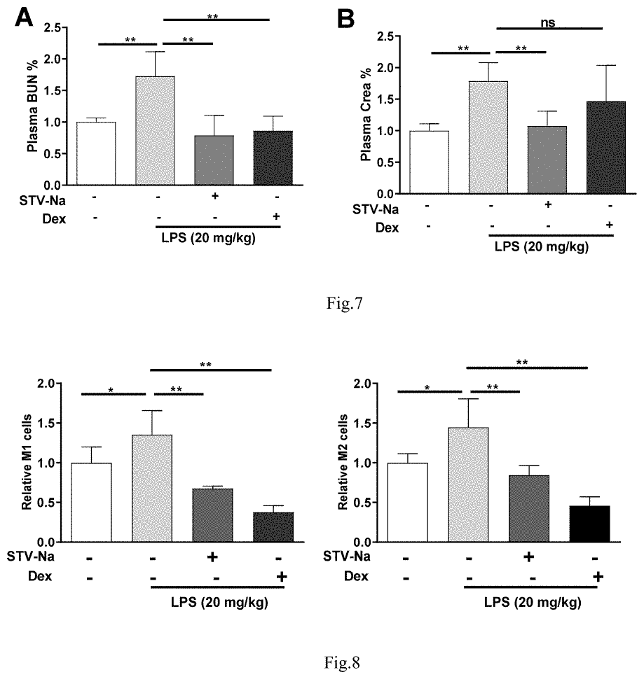

In order to observe the effect of compound A on the renal function of LPS-induced systemic inflammatory response in mice, we detected the changes of plasma BUN and Crea in mice, respectively. As shown in FIG. 7, after Compound A and Dex administration, the changes of BUN in the plasma of mice were significantly decreased when compared with the Control group (P<0.01). However, Crea level in plasma did not change significantly after Dex treatment, indicating that compound A can ameliorate renal function disorder of LPS-induced septic mice.

### EXAMPLE 9

This example illustrates the effect of compound A on macrophage polarization.
(1) Grouping of experimental animals: 6-8 week old Balb/c male mice were randomly divided into 4 groups after one week of adaptive feeding, 10 mice in each group, namely the Control group, the LPS group, the compound A group and the dexamethasone group. The LPS group, compound A group and dexamethasone group were given intraperitoneally injection of a 20 mg/kg of LPS. Samples are collected six hours after LPS injection.
(2) Extraction of primary peritoneal macrophages: Six hours after LPS injection, mice were sacrificed and the primary peritoneal macrophages were extracted.
(3) Flow cytometry for M1 and M2 macrophages: After blocked with MACS for 20 min on ice, cells are centrifuged at 1000 rpm, 4° C. for 5 min, and then discard the supernatant, add 0.2 μl of PE-anti-mouse F4/80 antibody and BV421-anti-CD11c antibody to the cell suspension, incubate on ice for 30 min in the dark, wash once with PBS, fix with 50 μl fixative solution on ice for 10 min, 50 μl of 1× permeabilization solution, 1000 rpm, centrifuge at 4° C. for 5 min, add 100 μl of 1× permeabilization solution to rupture the membrane, centrifuging, add 0.2 μl of FITC-anti-mouse CD206 antibody and incubate on ice for 30 min in the dark, Add 100 μl of permeabilization fluid and centrifuging, add 1 ml PBS to wash once, centrifuging, collecting sediment, resuspend in 200 μl PBS, FACSCelesta flow cytometry assay, FlowJo 7.6.1 software analyzes M1 (F4/80+CD11c+ CD206−) and M2 (F4/80+CD11c− CD206+).

In vivo, mouse peritoneal macrophages were used as the research object. Three days after the preventive administration of compound A, 20 mg/kg LPS was injected intraperitoneally for stimulation, and the mice were sacrificed 6 hours later to collect primary peritoneal macrophages for flow cytometry. As shown in FIG. 8, it was found that the M1 and M2 peritoneal macrophages were significantly reduced in the compound A and dexamethasone (the positive drug) group when compared with the LPS group. These indicate that compound A can regulate the M1/M2 macrophages ratio in LPS-induced septic mice thus maintaining the balance of M1/M2 macrophages in vivo.

The above-mentioned examples are only preferred embodiments of the present invention, but do not limit the invention in any way. It is obvious that all the modifications or rearrangements to these examples may be made by any technical persons skilled in art according to what have been disclosed by the invention. These modifications and rearrangements shall be included within the same scope of the invention.

1. The use of Kaurene compounds or its pharmaceutically acceptable salts thereof in a pharmaceutical preparation for preventing and treating sepsis or systemic inflammatory response syndrome (SIRS) and the multi-organ damage or failure caused by it.

2. The use of claim 1, wherein the disease of sepsis was induced by pathogens includes bacteria, viruses or fungi, and pathogen-associated molecular patterns (PAMPs) related to the abovementioned pathogens, include LPS, lipoproteins, glycoprotein, lipopeptides, nucleic acids, etc.

3. The use of claim 1, wherein the systemic inflammatory response syndrome (SIRS) is caused by non-pathogenic infections, including trauma, burns, myocardial infarction and heart failure, cerebral infarction, and inflammatory bowel disease, etc.

4. The use of claim 1, wherein the sepsis or systemic inflammatory response syndrome (SIRS) is characterized by the overproduction of systemic cytokines or cytokine storm.

5. The use of claim 1, wherein the multi-organ damage or failure is lung damage or failure, heart damage or failure, circulatory failure, liver damage or failure, kidney damage or failure, spleen damage or failure, or the combination of one or several of the above situation.

6. The use of claim 5, wherein the lung damage or failure is characterized by acute pulmonary depression, acute respiratory distress syndrome, or acute pulmonary failure.

7. The use of claim 5, wherein the heart damage or failure is characterized by cardiac dysfunction, heart failure or arrhythmia caused by sepsis or systemic inflammatory response syndrome (SIRS).

8. The use of claim 1, wherein the multi-organ damage or failure is characterized by organ fibrosis and remodeling caused by sepsis.

9. The use of claim 1, wherein the mechanism of action of the prevention and treatment involves inhibition of cytokine production or inhibition of cytokine storm. Cytokines include IFN-γ, TNF , IL-1β, IL-1, IL-6, IL-12, IL-13, IL-10, IL-23, IL-17 and IL-6, etc.

10. The use of claim 1, wherein the mechanism of action involves the inhibition and regulation of the activation and proliferation of macrophages.

11. The use of claim 1, wherein the mechanism of action involves the inhibition of activation and proliferation of inflammatory cells including leukocytes, neutrophils, monocytes and lymphocytes.

12. The use of claim 1, wherein the said compounds are represented by structural formula (I). Compounds of structural formula (I) may have one or more asymmetric centers, and may exist as different stereoisomers. 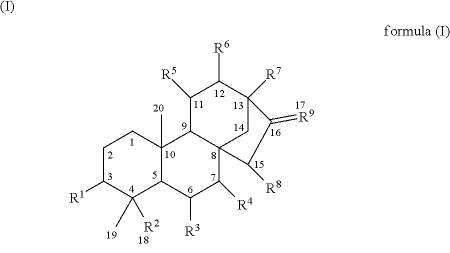
wherein ii. R1: Hydrogen, hydroxyl, or alkoxy.
iii. R2: Carboxyl, carboxylate, acyl halides, aldehyde, hydroxymethyl, and ester, acrylamide, acyl, or ether groups that can form carboxyl. which can generate carboxyl group.
iv. R3, R4, R5, R6, R8: Oxygen, hydroxyl, hydroxymethyl, and ester or alkoxymethyl
groups that hydrolyze to hydroxymethyl.
v. R7: Methyl, hydroxyl, and ester or alkoxymethyl groups that hydrolyze to hydroxymethyl. that can be hydrolyzed to hydroxymethyl. vi. R9: Methylene or oxygen.

13. The compound described in claim 12 is characterized by the fact that the structural formula (I) compound described therein is the compound shown in structural formula (II). The compound may have multiple asymmetric centers and have different stereoisomers or diastereoisomers. The absolute configurations of positions 8 and 13 are either (8R, 13S) or (8S, 13R). 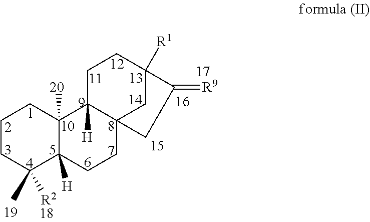
wherein vii. R2: Carboxylate, carboxylate, aldehyde group, hydroxymethyl, methyl ester, acyl methyl, acyl halide. viii. R7: Methyl, hydroxymethyl or methyl ether.
ix. R9: Methylene or oxygen.

14. The compound described in claim 12 is characterized by the fact that the structural formula (I) compound described therein is the compound shown in structural formula (A).

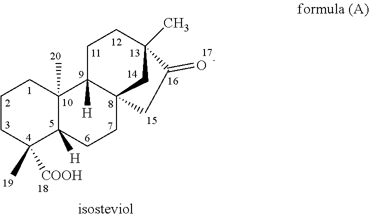

15. The compound described in claim 12 is characterized by the fact that the structural formula (I) compound described therein is the compound shown in structural formula (B).

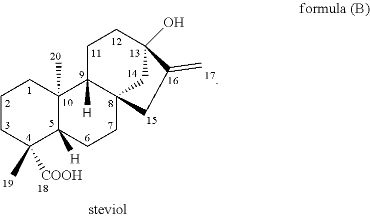

16. The use of claim 1, wherein said medicament are tablets, capsules, granules, suppositories, ointments, patches, injections, buccal tablets, chewables, and controlled release agents via oral, parenteral or implanted channels.

17. The use of claim 1, wherein said medicament is characterized by could be inhalant nebulizer, metered dose inhalant or dry powder inhalant via pulmonary or nasal delivery.

18. The use of claim 1, wherein said medicament is characterized by delivery to the patient in need through muscle, vein, abdominal cavity, interventional catheter and ventilator, using a standard medicinal liquid injection or infusion or other suitable dosage form.
